(19) United States
(12) Patent Application Publication
(10) Pub. No.: US 2022/0401018 A1
(43) Pub. Date: Dec. 22, 2022

(54) WAKE-UP DETECTION DEVICE

(71) Applicant: Xandar Kardian, Inc., New York, NY (US)

(72) Inventor: Sun Jong Yang, Gyeonggi-do (KR)

(73) Assignee: Xandar Kardian, Inc., New York, NY (US)

(21) Appl. No.: 17/772,333

(22) PCT Filed: Oct. 21, 2020
(86) PCT No.: PCT/KR2020/014413

§ 371 Date: Apr. 27, 2022

(30) Foreign Application Priority Data
Oct. 28, 2019 (KR) 10-2019-0134198

(51) Int. Cl. A61B 5/00; A61B 5/0205; A61B 5/11

(52) U.S. Cl. CPC A61B 5/4809; A61B 5/0205; A61B 5/1117; A61B 5/1115; A61B 5/1114; A61B 5/746; A61B 5/0002; A61B 5/02405

(57) ABSTRACT

The wake-up detection device may include a sensor configured to detect a movement of a person and a biosignal of the person; and a controller configured to analyze the movement and the biosignal recognized by the sensor, and determine whether the person wakes up from sleep, on the basis of a result of analyzing the movement and the biosignal. The controller may determine a change in a heart rate of the person when the person converts from a sleep state to a non-sleep state, and when it is determined that the change in the heart rate and the movement of the person increase, the controller may output a first alarm.

### CROSS-REFERENCE TO RELATED APPLICATION

The present application claims the benefit of and priority to Korean Patent Application No. 10-2019-0134198, filed on Oct. 28, 2019, the entire contents of which are incorporated herein by reference.

### BACKGROUND OF THE INVENTION

### Technical Field

The present invention relates to a wake-up detection device which detects a human wake-up by analyzing a human heart rate, a human movement and a human sleep pattern.

### Background Art

As interest in healthcare increases, interest in services that help the elderly or patients with reduced mobility to lead daily life with an easy mind is increasing. Since it is difficult for the elderly, the disabled and patients, living alone at home, to always be with their guardians, research on services for detecting sudden fall of the elderly or the disabled is ongoing. In particular, accidents in which the elderly or the disabled fall while moving after waking up from sleep during the night frequently occur. In addition, in the case of infants, falls often occur after waking up from sleep during the night. A fall, in which a human suddenly falls, refers to a phenomenon in which a human is injured by falling or coming down from a vertical state to a state parallel to the ground against the intention of the human. The fall may be a phenomenon which is even more fatal for the elderly or the disabled with degraded bodily functions.

In general, care facilities that protect the elderly or patients must periodically check the conditions of the elderly or patients. However, there are situations in which it is difficult for workers of the care facilities to grasp when an elderly person or a patient falls while waking up from sleep or moving during the night. As disclosed in Korean Patent No. 10-1860062, a device for detecting a fall of a human can detect a fall only after the human wakes up from a sleep state. A technique and a device for preventing a fall by monitoring the elderly or the disabled before they fall are not disclosed in a number of patent documents including Korean Patent No. 10-1860062. In addition, in the case of using a general technique, there is an inconvenience in that a sensor should be directly attached to the body in order to detect a biosignal or the movement of an elderly person or a patient during the night. Furthermore, in various cases such as a process of attaching the sensor or a state in which the battery of the sensor is discharged, there is a problem in that it is not possible to detect whether a resident has fallen.

### SUMMARY OF THE DISCLOSURE

It is an object of the present invention to provide a wake-up detection device capable of determining whether a human wakes up from sleep, by detecting a change in human heart rate and a change in a human movement.

It is an object of the present invention to provide a wake-up detection device capable of determining whether a human wakes up from sleep, by comparing whether a time at which a change in human heart rate and a change in human movement detected in real time occur matches a pre-stored sleep pattern.

It is an object of the present invention to provide a wake-up detection device capable of detecting a standing-up act and a falling act that may occur after a human wakes up from sleep and notifying a guardian thereof.

A wake-up detection device according to an embodiment of the present invention is provided. The wake-up detection device may include: a sensor configured to detect a movement of a person and a biosignal of the person; and a controller configured to analyze the movement and the biosignal recognized by the sensor, and determine whether the person wakes up from sleep, on the basis of a result of analyzing the movement and the biosignal, wherein the controller determines a change in a heart rate of the person when the person changes from a sleep state to a non-sleep state and wherein, when it is determined that the change in the heart rate and the movement of the person increase, the controller outputs a first alarm.

According to the exemplary embodiment, the controller may distinguish a first heart rate in a sleep state and a second heart rate when the person wakes up from sleep, and the controller may output the first alarm when it is determined that the heart rate of the person increases from the first heart rate to the second heart rate and the movement of the person increases beyond a preset range.

According to the exemplary embodiment, The preset range may mean an increase by a preset percentage on the basis of a movement of the person in a sleep state.

According to the exemplary embodiment, the controller may include a database which learns and stores information on a sleep pattern of the person, and the sleep pattern may include data on a time when the person wakes up from sleep and a time when the person stands up, for a preset period.

According to the exemplary embodiment, when a time at which the change in the heart rate and the movement of the person increase matches the sleep pattern stored in the database, the controller may output a second alarm.

According to the exemplary embodiment, when the sensor detects that the person has stood up at a sleeping place, the controller may output a third alarm.

According to the exemplary embodiment, when the sensor detects that the person has fallen in a space adjacent to the sleeping place, the controller may output a fourth alarm.

According to the exemplary embodiment, the controller may transmit the first alarm, the second alarm, the third alarm and the fourth alarm toward a terminal in a wireless communication scheme.

According to the exemplary embodiment, the sensor may detect a falling act of the person on the basis of at least one among a change in a distance between the person and the sleeping place, a change in a speed of a specific position of the sensed person, a change in an acceleration of the specific position of the sensed person and a change in a distance between the sensor and the person.

According to the exemplary embodiment, the controller may be automatically activated at a time at which the person enters a sleep state, by analyzing the sleep pattern of the person.

According to the exemplary embodiment, the controller may store data on a change in heart rate and a change in movement and a time at which the change in heart rate and the change in movement occur, in the database.

A wake-up detection device according to an embodiment of the present invention is provided. The wake-up detection device may include: a sensor configured to detect a movement of a person and a biosignal of the person; a database configured to learn and store information on a sleep pattern of the person; and a controller configured to analyze the movement, the biosignal and the sleep pattern recognized by the sensor, and determine whether the person wakes up from sleep, on the basis of a result of analyzing the movement, the biosignal and the sleep pattern, wherein the controller outputs alarms of a plurality of levels in consideration of at least two among a change in the heart rate of the person, a change in the movement of the person and the sleep pattern.

According to the exemplary embodiment, the change in the movement may include a first change meaning that the movement of the person increases beyond a preset range on the basis of a movement of the person who is in a sleep state, a second change meaning that it is detected that the person stands up, and a third change meaning that it is detected that the person falls after standing up.

According to the exemplary embodiment, the change in the heart rate may mean a change in the heart rate of the person when the person changes from a sleep state to a non-sleep state, and the sleep pattern may include data on a time when the person wakes up from sleep and a time when the person stands up, for a preset period.

According to the exemplary embodiment, when the first change and the change in the heart rate are detected, the controller may output a first alarm.

According to the exemplary embodiment, when the first change and the change in the heart rate are detected and a time at which the first change and the change in the heart rate are detected matches the sleep pattern, the controller may output a second alarm.

According to the exemplary embodiment, when the sensor detects the second change, the controller may output a third alarm.

According to the exemplary embodiment, when the sensor detects the third change, the controller may output a fourth alarm.

According to the embodiments of the present invention, the wake-up detection device may detect that a human in a sleep state wakes up during the night, and may notify the outside thereof. Accordingly, the wake-up detection device may prevent a patient, an infant and the elderly from being injured after waking up from sleep during the night.

According to the embodiments of the present invention, by changing the type of an alarm to be outputted, depending on a degree of wake-up, a guardian may clearly grasp a state of a human to be monitored.

### BRIEF DESCRIPTION OF THE DRAWINGS

The above and other features of the present disclosure are now be described in detail with reference to certain exemplary examples thereof illustrated in the accompanying drawings which are given herein below by way of illustration only, and thus are not limitative of the present disclosure, and wherein:

FIG. 1 is a view for explaining a situation in which a wake-up detection device according to an embodiment of the present invention is applied.

FIG. 2 is a block diagram illustrating the wake-up detection device according to the embodiment of the present invention.

FIG. 3 is a table showing levels of an alarm according to the embodiment of the present invention.

FIG. 4 is a view illustrating that the wake-up detection device according to the embodiment of the present invention detects that a person changes from a sleep state to a non-sleep state.

FIG. 5 is a view illustrating that the wake-up detection device according to the embodiment of the present invention detects a human standing-up act.

FIG. 6 is a view illustrating that the wake-up detection device according to the embodiment of the present invention detects a human falling act.

FIG. 7 is a flowchart illustrating a wake-up detection method according to an embodiment of the present invention.

### DETAILED DESCRIPTION

A wake-up detection device according to an embodiment of the present invention is provided. The wake-up detection device includes a sensor which detects a human movement and a human biosignal and a controller which analyzes the movement and the biosignal recognized by the sensor and determines whether a human wakes up from sleep, on the basis of a result of analyzing the movement and the biosignal, wherein the controller determines a change in human heart rate when the human changes from a sleep state to a non-sleep state and outputs a first alarm upon determining that the human heart rate changes and the human movement increases.

Advantages and features of the disclosure and methods for achieving the same will become more apparent after a reading of the following exemplary embodiments taken in conjunction with the drawings. The disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The disclosure will be defined only by the scope of the claims. Throughout the specification, like reference numerals refer to like components.

The term ‘ . . . section,’ ‘ . . . unit,’ ‘ . . . module’ or the like implies a unit for processing at least one function or operation, and may be implemented in hardware or software or in combination of hardware and software.

Moreover, the use of the terms first, second, etc. in the present specification is to distinguish one element from another, and is not limited to the order in the following description.

The present invention will be described in connection with what is presently considered to be practical exemplary embodiments. Although the exemplary embodiments of the present invention will be described, the present invention may be also used in various other combinations, modifications and environments. In other words, the present invention may be changed or modified within the range of concept of the invention disclosed in the specification, the range equivalent to the disclosure and/or the range of the technology or knowledge in the field to which the present invention pertains. The exemplary embodiments to be described below have been provided to explain the best state in carrying out the present invention. Therefore, they may be carried out in other states known to the field to which the present invention pertains in using other inventions such as the present invention and also be modified in various forms required in specific application fields and usages of the invention. Therefore, it is to be understood that the invention is not limited to the disclosed embodiments. It is to be understood that other embodiments are also included within the spirit and scope of the appended claims.

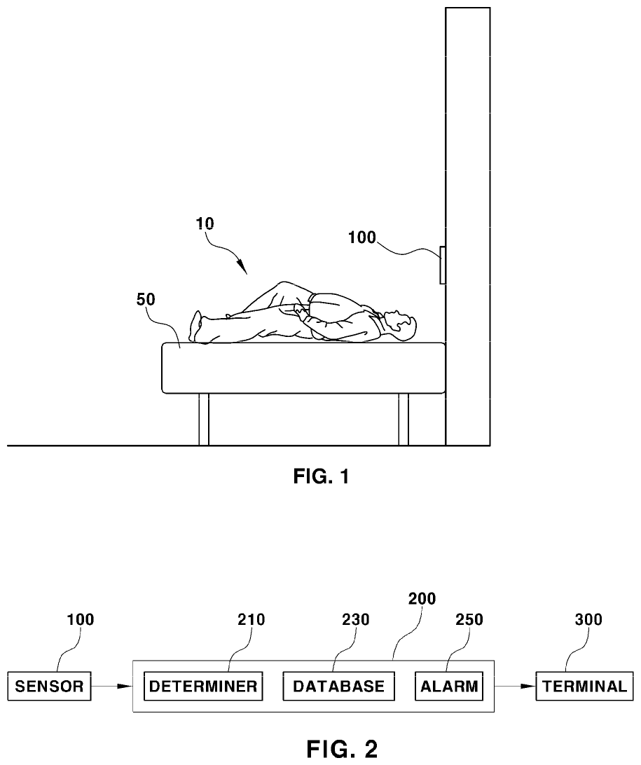

FIG. 1 is a view for explaining a situation in which a wake-up detection device according to an embodiment of the present invention is applied, and FIG. 2 is a block diagram illustrating the wake-up detection device according to the embodiment of the present invention.

Referring to FIGS. 1 and 2, a wake-up detection device 1 may determine whether a person 10 changes from a sleep state to a non-sleep state, by detecting a biosignal of the person 10 who is sleeping and a movement of the person 10. The wake-up detection device 1 may include a sensor 100, a controller 200 and a terminal 300.

The sensor 100 may detect a change in the biosignal of the person 10 who is sleeping in a sleeping place 50 and the movement of the person 10. The sensor 100 may be positioned in a place adjacent to the sleeping place 50. The sensor 100 may detect a change in the biosignal of the person 10 and the movement of the person 10 within a predetermined range on the basis of the sleeping place 50. For example, the sleeping place 50 may include a bed, a sofa or a floor. For example, the sensor 100 may be installed in a stand type or be attached to a wall, in the vicinity of the sleeping place 50. As the sensor 100, one sensor may be provided or a plurality of sensors may be provided.

For example, the sensor 100 may be any one of an impulse-radio ultra-wideband communication (IR-UWB) sensor, a LiDAR, a frequency-modulated continuous wave radar (FMCW radar) and a Doppler radar (Doppler radar). Ultra-wideband communication (UWB) refers to a radio technology which uses a frequency band of 500 MHz or more or in which a value defined as a fractional bandwidth is 25% or more. The fractional bandwidth means the bandwidth of a signal with respect to a center frequency. Ultra-wideband communication (UWB) as a radio technology which uses a wideband frequency has various advantages such as high distance resolution, transparency, strong immunity to narrowband noise and coexistence with other devices sharing a frequency. For example, ultra-wideband communication (UWB) has an advantage of being capable of detecting even a minute movement of an object with ultra-precise distance resolution characteristics for 1 cm or less.

An IR-UWB (impulse-radio ultra-wideband) radar (hereinafter, referred to as UWB radar) technology is a system which combines the ultra-wideband communication (UWB) technology with radar, and means a radar technology in which an impulse signal with wideband characteristics in a frequency domain and a very short duration is transmitted and a signal returning by being reflected from an object and a person is received to recognize a surrounding situation. The UWB radar system generates an impulse signal, having a time width of several nanoseconds to several picoseconds, in a signal generator and radiates the impulse signal at a wide angle or a narrow band angle through a transmitting antenna. The radiated signal is reflected by various objects or people in the environment, and the reflected signal may be converted into a digital signal through a receiving antenna and an analog-to-digital converter (ADC).

The LiDAR may emit a laser pulse, and may receive the laser pulse returned by being reflected from a surrounding target object to measure a distance from the LiDAR to the object. The LiDAR may detect not only a distance to the target object but also the speed of the target object, the shape of the target object and so forth, and furthermore, may be used to generate a three-dimensional image of the surroundings.

The biosignal may include at least one of the heart rate and the respiration of the person 10. Preferably, the biosignal may be the heart rate of the person 10. The sensor 100 may receive a signal reflected by the person 10 to detect the movement of the chest or stomach of the person 10, and through this, may detect the heart rate or the respiration of the person 10.

The sensor 100 may detect a shortest distance to the person 10. The sensor 100 may detect a shortest distance to the person 10 on the basis of a specific position on the person 10. For example, the specific position may mean a portion of the body part of the person 10 such as the head, torso and legs of the person 10, and may mean a position from which a shortest distance from the sensor 100 to the person 10 is obtained. Therefore, the specific position may mean a position that is changed in real time according to the movement of the person 10.

The sensor 100 may determine the standing-up and falling acts of the person 10 on the basis of the shortest distance to the person 10. Optimal information for determining the standing-up and falling acts may be the height of a specific position on the person 10. For example, when the person 10 stands up, the sensor 100 may detect the torso of the person 10. When the person 10 stands up, the distance from the torso of the person 10 to the sensor 100 may be a shortest distance. On the other hand, when the person 10 falls, various parts such as the torso, head and legs of the person 10 may be defined as a specific position according to a direction in which the person 10 falls.

The sensor 100 may detect a falling act of the person 10 on the basis of a sudden change in a specific position of the person 10 and a speed change and an acceleration change of the specific position of the person 10.

The controller 200 may include a determiner 210, a database 230 and an alarm 250. The determiner 210, the database 230 and the alarm 250 may be components which are defined according to the functions of the controller 200.

The determiner 210 may detect whether the person 10 is in a sleep state or a non-sleep state, through a change in the respiration of the person 10, a change in the movement of the heart, a change in the heart rate and a change in the movement of the person 10. The determiner 210 may analyze the movement of the person 10 and the biosignal of the person 10 recognized by the sensor 100, and may determine whether the person 10 wakes up from sleep, on the basis of a result of analyzing the movement and the biosignal. The change in the movement may include a first change meaning that the movement of the person 10 increases beyond a preset range on the basis of a movement of the person 10 who is in a sleep state, a second change meaning that it is detected that the person 10 stands up, and a third change meaning that it is detected that the person 10 falls after standing up.

For example, the determiner 210 may determine whether the person 10 wakes up from sleep, on the basis of a change in the heart rate of the person 10 and a change in the movement of the person 10 when the person 10 changes from a sleeping state to a non-sleep state. For example, the heart rate of the person 10 in a sleep state may be 50 BPM to 60 BPM, and the heart rate of the person 10 when the person 10 changes from a sleep state to a non-sleep state may be 65 BPM to 70 BPM. However, since a heart rate in a sleep state and a heart rate when a person changes from a sleep state to a non-sleep state may vary depending on the person, the controller 200 may store or learn in advance the heart rate of the person 10 in a sleep state. The heart rate of the person 10 when the person 10 changes from a sleep state to a non-sleep state may be increased by a preset percentage compared to the heart rate of the person 10 in a sleep state. For example, a heart rate when a sleep state changes to a non-sleep state may be a heart rate that is increased by about 10% compared to a heart rate in a sleep state. However, the preset percentage may be changed by a user or may be changed on the basis of data learned by the controller 200.

For example, when the movement of the person 10 in a sleep state increases beyond the preset range, the determiner 210 may determine whether the person 10 wakes up from sleep (the first change in movement). That is to say, when the movement of the person 10 increases by a predetermined level on the basis of the data of the sensor 100 having detected a change in the movement of the person 10, the determiner 210 may determine that the person 10 is changing from a sleep state to a non-sleep state. The preset range may mean an increase by a preset percentage on the basis of a movement of a person in a sleep state. For example, the preset range may be about 20%. However, since a movement in a sleep state and a movement when a person changes from a sleep state to a non-sleep state may vary depending on the person, the controller 200 may store or learn in advance the degree of the movement of the person 10 in a sleep state.

On the basis of the data sensed by the sensor 100, the determiner 210 may determine whether the person 10 stands up at the sleeping place 50 (the second change in movement). Standing-up of the person 10 may have a meaning including both an act of sitting while lying at the sleeping place 50 and an act of standing up completely.

On the basis of the data sensed by the sensor 100, the determiner 210 may determine whether the person 10 falls after standing up at the sleeping place 50 (the third change in movement).

The determiner 210 may set a Cartesian coordinate system which has one axis facing a plurality of sensors 100. The determiner 210 may set the coordinates of the sensors 100 so that the other two coordinates except coordinates on the direction in which the sensors 100 are disposed are the same. In other words, the sensors 100 may be set to be disposed at the same coordinates except the coordinates on the one axis. Therefore, by combining information on shortest distances to the person 10 measured by the respective sensors 100 and information on distances between the sensors 100, the determiner 210 may calculate the height of the person 10. Since the determiner 210 receives in real time information on shortest distances detected by the sensors 100, it is possible to continuously monitor a change in the height of the person 10. Accordingly, when the height of the person 10 suddenly decreases, the determiner 210 may determine that the person 10 has fallen, and when the height of the person 10 suddenly increases, the determiner 210 may determine that the person 10 has stood up. In this case, the determiner 210 may consider the relationship between the ground or the sleeping place 50 and the height of the person 10.

Unlike the above-described example, the detection of the standing-up and falling acts of the person 10 by the determiner 210 may not be limited to the above-described method.

The database 230 may store and learn data on the heart rate of the person 10 in a sleep state and the heart rate of the person 10 when the person 10 changes from a sleep state to a non-sleep state. A heart rate in a sleep state and a heart rate when a person changes from a sleep state to a non-sleep state may vary from person to person. Accordingly, depending on a user, the database 230 may differently store a heart rate criterion for determining a sleep state and a non-sleep state.

The database 230 may store and learn data on the movement of the person 10 in a sleep state and the movement of the person 10 when the person 10 changes from a sleep state to a non-sleep state. A movement in a sleep state and a movement when a person changes from a sleep state to a non-sleep state may vary from person to person. Accordingly, depending on a user, the database 230 may differently store a movement change criterion for determining a sleep state and a non-sleep state.

The database 230 may learn and store information on the sleep pattern of the person 10. The sleep pattern may include data on a time when a person wakes up from sleep and a time when a person stands up, for a preset period. The database 230 may store data obtained by monitoring the sleep pattern of the person 10 for a preset period. Accordingly, the database 230 may store various data on a frequency at which the person 10 wakes up from sleep during sleeping, when the person 10 wakes up from sleep in general in the case where the person 10 wakes up from sleep during sleeping, a frequency at which the person 10 stands up after waking up from sleep, a time at which the person 10 stands up after waking up from sleep, and so forth. Moreover, the database 230 may store data on a change in heart rate and a change in movement and a time at which a change in heart rate and a change in movement occur. The determiner 210 may determine whether a time at which a change in heart rate and a change in movement of the person 10 are detected in real time matches the sleep pattern stored in the database 230. When the time at which the change in heart rate and the change in movement of the person 10 are detected in real time matches the sleep pattern stored in the database 230, the determiner 210 may determine that the data detected by the sensor 100 has high reliability. For example, the sleep pattern stored in the database 230 may indicate that the person 10 frequently wakes up from sleep between 3:00 and 4:00 in the morning. When the determiner 210 determines, as a result of analyzing the change in heart rate and the change in movement of the person 10 detected by the sensor 100, that the person 10 is changing from a sleep state to a non-sleep state, the determiner 210 may determine that the determination that the person 10 has woken up from sleep has high reliability.

The determiner 210 may set an activation time of the wake-up detection device 1 by using the data on the sleep pattern of the person 10 stored in the database 230. Specifically, the wake-up detection device 1 may be automatically activated on the basis of data on a time at which the person 10 enters a sleep state.

The alarm 250 may output an alarm of a plurality of levels in consideration of at least two factors among a change in the biosignal of the person 10 (in particular, a change in heart rate), a change in the movement of the person and a sleep pattern. The determiner 210 may set a plurality of wake-up stages on the basis of the state of the person 10. For example, in a first wake-up stage, the determiner 210 determines a non-sleep state on the basis of a change in heart rate and a change in movement. In a second wake-up stage, the determiner 210 determines a non-sleep state on the basis of whether a change in heart rate, a change in movement and a sleep pattern match changes in heart rate and movement detected in real time. In a third wake-up stage, the determiner 210 determines a non-sleep state on the basis of that standing-up of the person 10 is detected, and in a fourth wake-up stage, the determiner 210 determines a non-sleep state on the basis of that a falling act is detected after the standing-up of the person 10. The fourth wake-up stage is to notify the outside that the person 10 falls after waking up from sleep, and may be a stage for determining that a baby, a patient, the elderly, etc. are in a dangerous state at dawn. The alarm 250 may output the alarm of the plurality of levels on the basis of the wake-up stages determined by the determiner 210. For example, the alarm 250 may output a first alarm at the first wake-up stage, may output a second alarm at the second wake-up stage, may output a third alarm at the third wake-up stage, and may output a fourth alarm at the fourth wake-up stage. The alarm outputted by the alarm 250 may be transmitted to the terminal 300 or may be outputted as voice. The alarm 250 may transmit the first alarm, the second alarm, the third alarm and the fourth alarm toward the terminal 300 in a wireless communication scheme. For example, the alarm transmitted to the terminal 300 may be displayed on the display of the terminal 300 or may be outputted as a voice through the speaker of the terminal 300.

According to the embodiment of the present invention, the wake-up detection device 1 may detect that the person 10 in a sleep state wakes up during the night, and may notify the outside thereof. Accordingly, the wake-up detection device 1 may prevent a patient, an infant and the elderly from being injured after waking up from sleep during the night.

According to the embodiment of the present invention, by sequentially outputting alarms of several levels, the wake-up detection device 1 may sequentially notify a person protecting or observing the person 10 of a current state of the person 10. Accordingly, a guardian may prevent an accident that may occur to the person 10.

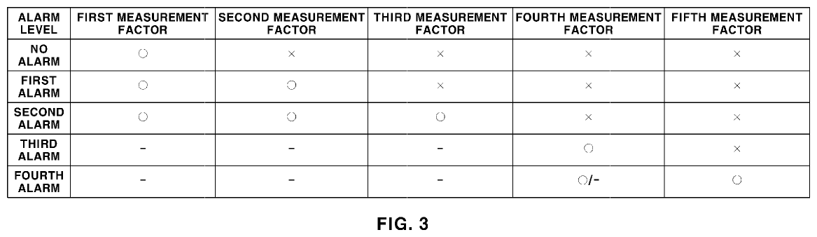

FIG. 3 is a table showing levels of an alarm according to the embodiment of the present invention.

Referring to FIGS. 2 and 3, five measurement factors may be used to determine whether a person changes from a sleep state to a non-sleep state and whether there is a risk of injury to the person. The measurement factors may include a first measurement factor, a second measurement factor, a third measurement factor, a fourth measurement factor and a fifth measurement factor.

The first measurement factor may be a change in the heart rate of a person. Specifically, the controller 200 may distinguish a first heart rate in a sleep state and a second heart rate when waking up from sleep, and the first measurement factor may mean that the heart rate of the person rises from the first heart rate to the second heart rate.

The second measurement factor may be a change in the movement of the person. Specifically, the controller 200 may distinguish a first movement in a sleep state and a second movement when waking up from sleep, and the second measurement factor may mean that the degree of the movement of the person changes from the first movement to the second movement. The second movement may mean that the movement increases by a preset percentage compared to the first movement.

The third measurement factor may mean whether a time at which a change in the heart rate and a change in the movement of the person occur matches a sleep pattern stored in the database 230. Specifically, the third measurement factor may mean a case where a time at which a change in the heart rate and a change in the movement of the person detected in real time occur matches a time pattern, stored in the database 230, with which the person frequently wakes up during the night.

The fourth measurement factor may mean that the standing-up act of the person is detected. The standing-up act of the person means that the person stands up after the person enters a sleep state, and may be interpreted as including both sitting at a sleeping place and completely standing up.

The fifth measurement factor may mean that the falling act of the person is detected. The falling act of the person may mean that the person falls after standing up.

The controller 200 may output the first alarm when the data sensed by the sensor 100 satisfies both the first measurement factor and the second measurement factor.

The controller 200 may output the second alarm when the data sensed by the sensor 100 satisfies all of the first measurement factor, the second measurement factor and the third measurement factor.

The controller 200 may output the third alarm when the data sensed by the sensor 100 satisfies the fourth measurement factor.

The controller 200 may output the fourth alarm when the data sensed by the sensor 100 satisfies the fifth measurement factor. The controller 200 may also be designed to output the fourth alarm when both the fourth measurement factor and the fifth measurement factor are satisfied.

According to the embodiment of the present invention, by changing the type of an alarm to be outputted, depending on a degree of wake-up, a guardian may clearly grasp a state of a person to be monitored.

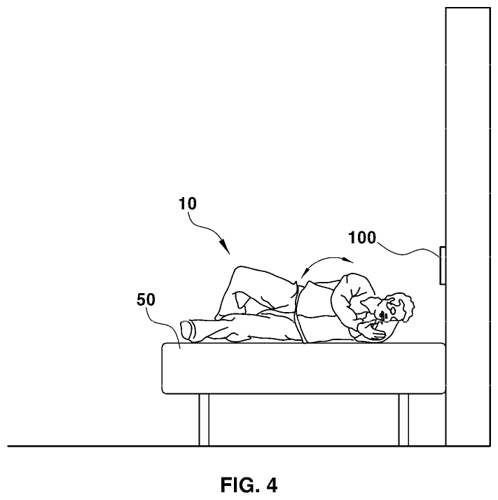

FIG. 4 is a view illustrating that the wake-up detection device according to the embodiment of the present invention detects that a person changes from a sleep state to a non-sleep state.

Referring to FIGS. 2 to 4, the person 10 may minutely move at the sleeping place 50, and the heart rate of the person 10 may slightly increase in a sleep state. The sensor 100 of the wake-up detection device 1 may be disposed in a space adjacent to the sleeping place 50 to detect a change in the heart rate and a change in the movement of the person 10. The determiner 210 may determine whether the data sensed by the sensor 100 satisfies both the first measurement factor and the second measurement factor.

For example, when the heart rate of the person 10 changes from a reference heart rate of a sleep state to a reference heart rate of a non-sleep state and the degree of the movement of the person 10 changes from a reference movement of a sleep state to a reference movement of a non-sleep state, the determiner 210 may determine that the person is changing from a sleep state to a non-sleep state. Namely, the determiner 210 may determine that the person 10 has woken up from sleep or is immediately before waking up from sleep. At this time, the alarm 250 may output the first alarm.

The determiner 210 may determine whether the data sensed by the sensor 100 satisfies all of the first measurement factor, the second measurement factor and the third measurement factor.

For example, when the heart rate of the person 10 changes from a reference heart rate of a sleep state to a reference heart rate of a non-sleep state and the degree of the movement of the person 10 changes from a reference movement of a sleep state to a reference movement of a non-sleep state, the determiner 210 may determine that the person is changing from a sleep state to a non-sleep state. Furthermore, when a time at which the change in the heart rate and the change in the movement of the person 10 occur matches the sleep pattern stored in the database 230, the determiner 210 may determine that the person 10 has woken up from sleep or is about to wake up from sleep. That is to say, the determiner 210 may determine that a situation in which the first measurement factor, the second measurement factor and the third measurement factor are satisfied is a situation with higher reliability than a situation in which the first measurement factor and the second measurement factor are satisfied. The situation with higher reliability may mean that a situation in which the person 10 has woken up from sleep or is about to wake up from sleep has higher reliability. At this time, the alarm 250 may output the second alarm.

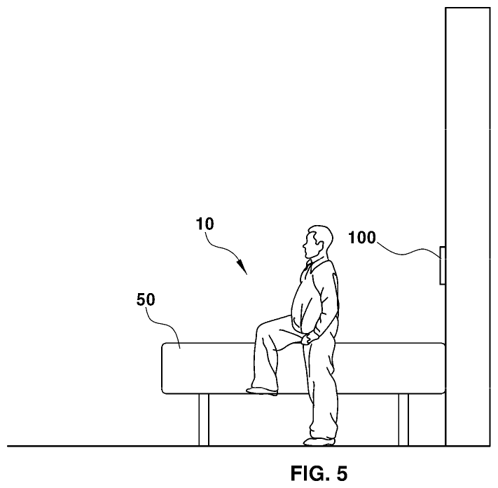

FIG. 5 is a view illustrating that the wake-up detection device according to the embodiment of the present invention detects a human standing-up act.

Referring to FIGS. 2, 3 and 5, the person 10 may stand up at the sleeping place 50 after waking up from sleep. In the present embodiment, the person 10 sits on a bed, and the sensor 100 may detect the person 10 who sits on the bed. The determiner 210 may determine whether the data sensed by the sensor 100 satisfies the fourth measurement factor. At this time, the alarm 250 may output the third alarm.

For example, the sensor 100 may continuously measure the shortest distance between the person 10 and the sensor 100, and the determiner 210 may monitor whether the shortest distance between the person 10 and the sensor 100 changes. The determiner 210 may determine whether the person 10 has stood up, on the basis of whether the shortest distance between the person 10 and the sensor 100 changes, whether the person 10 is detected at a position where the sensor 100 is disposed, whether the distance between the sleeping place and a specific position of the person 10 changes and whether the speed and acceleration of the specific position of the person 10 change. For example, when the height of the person 10 suddenly increases, the determiner 210 may determine that the person 10 has stood up.

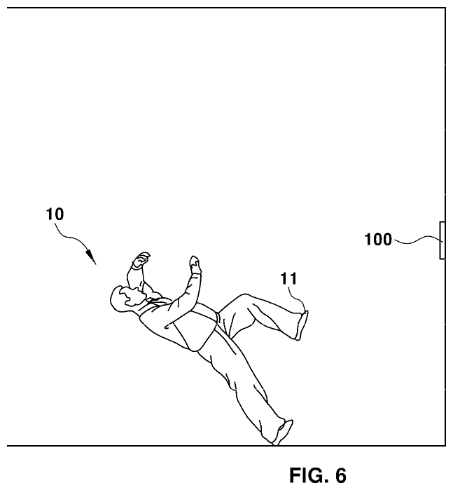

FIG. 6 is a view illustrating that the wake-up detection device according to the embodiment of the present invention detects a human falling act.

Referring to FIGS. 2, 3 and 6, the person 10 may fall after standing up. A patient, an infant and the elderly often fall after waking up from sleep. The sensor 100 may continuously sense the person 10 to grasp whether there is a falling act of the person 10 after standing up. The determiner 210 may determine whether the data sensed by the sensor 100 satisfies the fifth measurement factor. At this time, the alarm 250 may output the fourth alarm.

For example, the sensor 100 may continuously measure the shortest distance between the person 10 and the sensor 100, and the determiner 210 may monitor whether the shortest distance between the person 10 and the sensor 100 changes. The determiner 210 may determine whether the person 10 has fallen, on the basis of whether the shortest distance between the person 10 and the sensor 100 changes, whether the person 10 is detected at a position where the sensor 100 is disposed, whether the distance between the sleeping place 50 and a specific position 11 of the person 10 changes and whether the speed and acceleration of the specific position 11 of the person 10 change. For example, when the height of the person 10 suddenly decreases, the determiner 210 may determine that the person 10 has fallen, and when the speed or acceleration of the specific position 11 of the person 10 suddenly changes, the determiner 210 may determine that the person 10 has fallen.

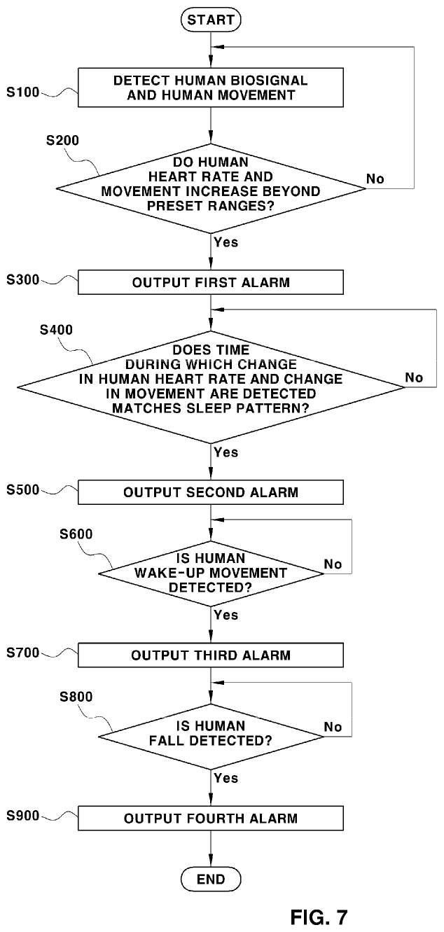

FIG. 7 is a flowchart illustrating a wake-up detection method according to an embodiment of the present invention.

Referring to FIG. 7, a sensor may detect a human biosignal and a human movement. The biosignal may include a person's respiration, heart rate and chest movement. Preferably, the biosignal may be a human heart rate (S100).

The sensor may detect a change in the heart rate of the person. Specifically, the sensor may detect a change from a reference heart rate in a sleep state to a reference heart rate in a non-sleep state. In addition, the sensor may detect a change in the degree of movement from a reference movement in a sleep state to a reference movement in a non-sleep state (S200).

A controller may determine whether the person has woken up from sleep, on the basis of the data measured by the sensor. When it is detected that a reference heart rate in a sleep state changes to a reference heart rate in a non-sleep state and the degree of movement changes from a reference movement in a sleep state to a reference movement in a non-sleep state, the controller may determine that the person has woken up from sleep. Accordingly, the controller may output a first alarm to notify that the person has woken up from sleep. When the controller determines that the person maintains a sleep state even though there are changes in heart rate and movement, the sensor may continuously detect a human biosignal and a human movement (S300).

After the first alarm is outputted, the controller may determine whether a time at which a change in the heart rate and a change in the movement of the person measured by the sensor are detected matches a sleep pattern stored in a database. Information on the sleep pattern may be learned and stored. The sleep pattern may include data on a time when a person wakes up from sleep and a time when a person stands up, for a preset period (S400).

When the time at which the change in the heart rate and the change in the movement of the person measured by the sensor are detected matches the sleep pattern stored in the database, the controller may output a second alarm. In other words, when the time at which the change in the heart rate and the change in the movement of the person measured by the sensor are detected matches the sleep pattern stored in the database, the controller may determine that the probability for the person to have woken up from sleep is higher than the case where only the change in the heart rate and the change in the movement of the person are detected. When the time at which the change in the heart rate and the change in the movement of the person measured by the sensor are detected does not match the sleep pattern stored in the database, the controller may continuously determine whether a time at which a change in the heart rate and a change in the movement of the person measured by the sensor are detected matches the sleep pattern (S500).

The sensor may detect the standing-up act of the person. The standing-up act of the person may be determined by the controller on the basis of the data sensed by the sensor (S600).

When the controller determines that the standing-up act of the person has occurred, the controller may output a third alarm. When the standing-up act of the person is not detected, the sensor may continuously detect the movement of the person, and the controller may continuously analyze the movement of the person by analyzing the data sensed by the sensor (S700).

The sensor may detect the falling act of the person. The falling act may mean that the person falls after the standing-up act of the person. The falling act of the person may be determined by the controller on the basis of the data sensed by the sensor (S800).

When the controller determines that the falling act of the person has occurred, the controller may output a fourth alarm. When the falling act of the person is not detected, the sensor may continuously detect the movement of the person, and the controller may continuously analyze the movement of the person by analyzing the data sensed by the sensor (S900).

The exemplary embodiments of the present disclosure have been described above with reference to the accompanying drawings, but those skilled in the art will understand that the present disclosure may be implemented in another particular form without changing the technical spirit or an essential feature thereof. Therefore, it should be understood that the embodiments described above are exemplary in all respects and not restrictive.

What is claimed is:

1. A wake-up detection device comprising:
a sensor configured to detect a movement of a person and a biosignal of the person; and
a controller configured to analyze the movement and the biosignal recognized by the sensor, and determine whether the person wakes up from sleep, on the basis of a result of analyzing the movement and the biosignal,
wherein the controller determines a change in a heart rate of the person when the person changes from a sleep state to a non-sleep state,
wherein, when it is determined that the change in the heart rate and the movement of the person increase, the controller outputs a first alarm,
wherein the controller distinguishes a first heart rate in a sleep state and a second heart rate when the person wakes up from sleep, and
wherein the controller outputs the first alarm when it is determined that the heart rate of the person increases from the first heart rate to the second heart rate and the movement of the person increases beyond a preset range.

2. The wake-up detection device according to claim 1, wherein the preset range means an increase by a preset percentage on the basis of a movement of the person in a sleep state.

3. The wake-up detection device according to claim 1, wherein
the controller includes a database which learns and stores information on a sleep pattern of the person, and
the sleep pattern includes data on a time when the person wakes up from sleep and a time when the person stands up, for a preset period.

4. The wake-up detection device according to claim 3, wherein, when a time at which the change in the heart rate and the movement of the person increase matches the sleep pattern stored in the database, the controller outputs a second alarm.

5. The wake-up detection device according to claim 4, wherein, when the sensor detects that the person has stood up at a sleeping place, the controller outputs a third alarm.

6. The wake-up detection device according to claim 5, wherein, when the sensor detects that the person has fallen in a space adjacent to the sleeping place, the controller outputs a fourth alarm.

7. The wake-up detection device according to claim 6, wherein the controller transmits the first alarm, the second alarm, the third alarm and the fourth alarm toward a terminal in a wireless communication scheme.

8. The wake-up detection device according to claim 6, wherein the sensor detects a falling act of the person on the basis of at least one among a change in a distance between the person and the sleeping place, a change in a speed of a specific position of the sensed person, a change in an acceleration of the specific position of the sensed person and a change in a distance between the sensor and the person.

9. The wake-up detection device according to claim 1, wherein the controller is automatically activated at a time at which the person enters a sleep state, by analyzing the sleep pattern of the person.

10. The wake-up detection device according to claim 1, wherein the controller stores data on a change in heart rate and a change in movement and a time at which the change in heart rate and the change in movement occur, in the database.

11. A wake-up detection device comprising:
a sensor configured to detect a movement of a person and a biosignal of the person;
a database configured to learn and store information on a sleep pattern of the person; and
a controller configured to analyze the movement, the biosignal and the sleep pattern recognized by the sensor, and determine whether the person wakes up from sleep, on the basis of a result of analyzing the movement, the biosignal and the sleep pattern,
wherein the controller outputs alarms of a plurality of levels in consideration of at least two among a change in the heart rate of the person, a change in the movement of the person and the sleep pattern.

12. The wake-up detection device according to claim 11, wherein the change in the movement includes a first change meaning that the movement of the person increases beyond a preset range on the basis of a movement of the person who is in a sleep state, a second change meaning that it is detected that the person stands up, and a third change meaning that it is detected that the person falls after standing up.

13. The wake-up detection device according to claim 12, wherein
the change in the heart rate means a change in the heart rate of the person when the person changes from a sleep state to a non-sleep state, and
the sleep pattern includes data on a time when the person wakes up from sleep and a time when the person stands up, for a preset period.

14. The wake-up detection device according to claim 13, wherein, when the first change and the change in the heart rate are detected, the controller outputs a first alarm.

15. The wake-up detection device according to claim 13, wherein, when the first change and the change in the heart rate are detected and a time at which the first change and the change in the heart rate are detected matches the sleep pattern, the controller outputs a second alarm.

16. The wake-up detection device according to claim 13, wherein, when the sensor detects the second change, the controller outputs a third alarm.

17. The wake-up detection device according to claim 13, wherein, when the sensor detects the third change, the controller outputs a fourth alarm.
